(19) United States
(12) Patent Application Publication
(10) Pub. No.: US 2025/0345051 A1
(43) Pub. Date: Nov. 13, 2025

(54) SEPTAL OCCLUDER WITH GATE

(71) Applicant: EDWARDS LIFESCIENCES CORPORATION, Irvine, CA (US)

(72) Inventors: Natan Anatoly Bukhdruker, Tirat Carmel (IL); Yaron Herman, Givat Ada (IL)

(21) Appl. No.: 19/277,364

(22) Filed: Jul. 22, 2025

Related U.S. Application Data
(63) Continuation of application No. PCT/US2024/012631, filed on Jan. 23, 2024.
(60) Provisional application No. 63/481,983, filed on Jan. 27, 2023.

(51) Int. Cl. A61B 17/00

(52) U.S. Cl. CPC A61B 17/0057; A61B 2017/00592; A61B 2017/00597; A61B 2017/00606; A61B 2017/00623

(57) ABSTRACT

A septal occluder includes a structural framework and a flexible skin supported by the structural framework. The structural framework includes a cylindrical section interposed between end sections. Each of the cylindrical section and end sections include interconnected struts that form a mesh. The cylindrical section extends along a centerline axis to define a bore. The flexible skin includes a membrane gate, a mechanical gate, or a membrane gate and mechanical gate spanning the bore.

### CROSS-REFERENCE TO RELATED APPLICATION(S)

This application is a continuation of International Application No. PCT/US2024/012631, filed Jan. 23, 2024, which claims the benefit of U.S. Provisional Application No. 63/481,983, filed Jan. 27, 2023, the disclosures of which are hereby incorporated by reference in their entireties.

### BACKGROUND

The present disclosure relates generally to implantable devices and, more specifically, to occluding devices that are implantable into a patient's inter-atrial septum.

Practitioners use a septal occluder to close an opening through a patient's inter-atrial septum, blocking a path between the right atrium and the left atrium of the patient's heart. Openings through the inter-atrial septum can be heart defects, such as a patent foramen ovale, or can result from surgical procedures that require transseptal puncture, such as left heart electrophysiology ablations, percutaneous mitral valve repair/implantation, left atrial appendage occlusion, paraprosthetic valve leak repair, and left ventricular assist device positioning among other possible procedures. Once a defect or a surgically-created opening in the patient's inter-atrial septum is closed with a septal occluder, subsequent transseptal punctures cannot utilize the same puncture site because the structure of conventional septal occluders obstructs the transseptal puncture. Most procedures that require transseptal puncture cannot be performed at a different puncture site, rendering subsequent procedures inadvisable.

### SUMMARY

A septal occluder in accordance with an example of this disclosure includes a flexible skin attached to a structural framework. The structural framework includes a cylindrical section interposed between end sections that extends along a centerline axis to define a bore. Each end section extends radially outward relative to the centerline axis. The flexible skin includes a membrane gate spanning the bore.

A system in accordance with another example of this disclosure includes a catheter and a septal occluder. The catheter includes a sheath and an inner tube. The septal occluder includes a flexible skin attached to a structural framework. The structural framework includes a cylindrical section interposed between end sections that extends along a centerline axis to define a bore. Each end section extends radially outward relative to the centerline axis. The flexible skin includes a membrane gate spanning the bore. The septal occluder is retained within the sheath.

A method of implanting a septal occluder in accordance with an example of this disclosure includes guiding a catheter through an opening in an inter-atrial septum. The method further includes retracting a sheath of the catheter to expose a first fixation wall of a septal occluder retained within the sheath of the catheter and withdrawing the catheter until the first fixation wall abuts the inter-atrial septum. The method further includes retracting the sheath to expose a second fixation wall of the septal occluder and removing the catheter while the septal occluder remains engaged to the inter-atrial septum.

A method of recrossing a septal occluder in accordance with another example of this disclosure includes guiding a catheter into a right atrium of a patient's heart, which contains a septal occluder implanted within an opening of the inter-atrial septum. The method further includes extending a needle from within the catheter to pierce a membrane gate of the septal occluder.

### BRIEF DESCRIPTION OF THE DRAWINGS

FIG. 1 is a schematic diagram of a heart and vasculature.

FIG. 2 is a schematic cross-sectional view of the heart.

FIG. 3A is an isometric view of a septal occluder that includes membrane gates.

FIG. 3B is an isometric view of the septal occluder with a portion of the flexible skin removed to show a structural framework.

FIG. 3C is a side view of the septal occluder with half of the flexible skin removed to show the structural framework of the septal occluder.

FIG. 3D is an end view of the septal occluder with an end face of the skin removed to reveal an end of the structural framework.

FIG. 4A is a cross-sectional view of the septal occluder implanted in a patient's heart prior to piercing the membrane gates.

FIG. 4B is a cross-sectional view of the septal occluder implanted in a patient's heart with pierced membrane gates.

FIG. 5A is an isometric cross-sectional view of a septal occluder that includes a mechanical gate in a closed position.

FIG. 5B is an isometric cross-sectional view of the septal occluder that includes a mechanical gate in an open position.

FIG. 5C is an isometric view of a mechanical gate in an open position.

FIG. 6A is a cross-sectional view of a septal occluder with membrane gates and a mechanical gate implanted in a patient's heart with the mechanical gate in a closed position.

FIG. 6B is a cross-sectional view of the septal occluder with membrane gates and a mechanical gate implanted in a patient's heart with the mechanical gate in an open position.

FIGS. 7A-7C are schematic views of a distal end of a delivery catheter that retains a septal occluder.

FIGS. 8A-8E are schematic views depicting the implantation sequence of a septal occluder into a patient's heart.

FIG. 9 is a flowchart describing a method of implanting a septal occluder in a patient's heart.

FIGS. 10A-10E are schematic views depicting the recrossing sequence of the septal occluder.

FIG. 11 is a flowchart describing a method of recrossing a septal occluder equipped with a gate.

### DETAILED DESCRIPTION

### Anatomy of Heart H and Vasculature V

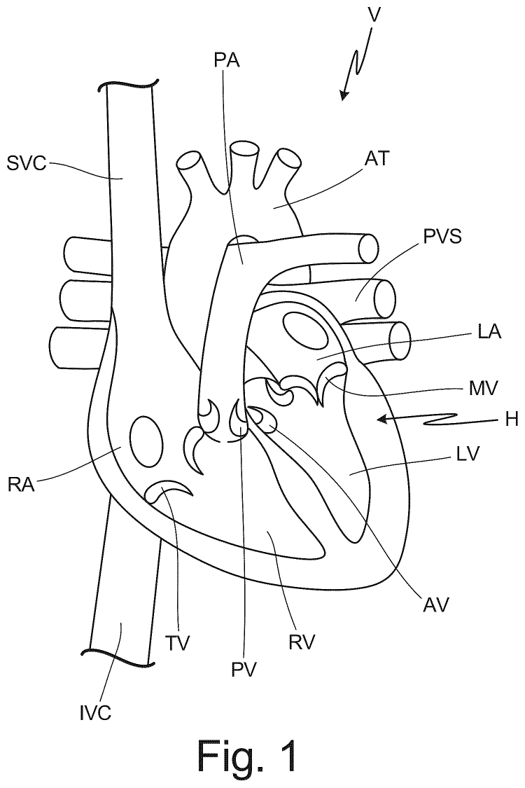

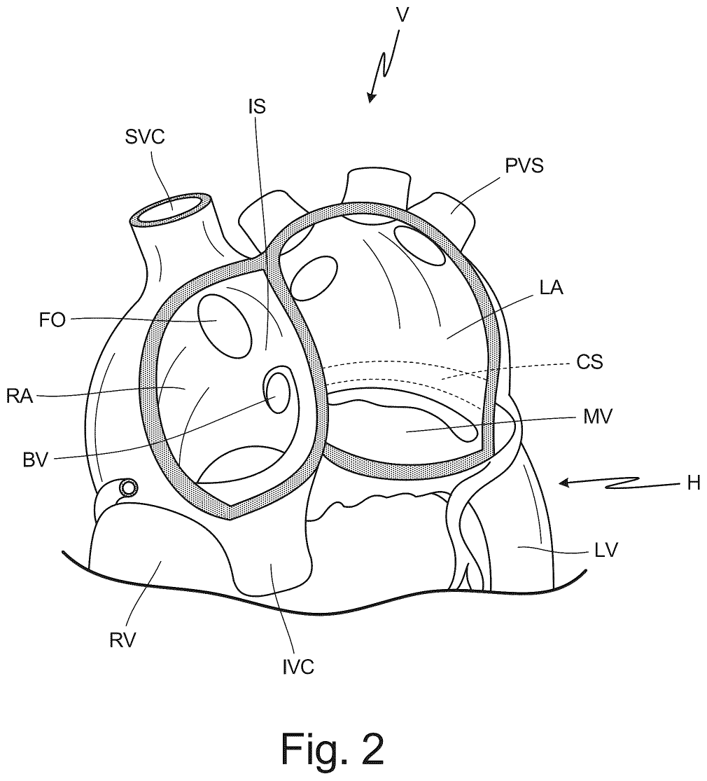

FIG. 1 is a schematic diagram of heart H and vasculature V. FIG. 2 is a cross-sectional view of heart H. FIGS. 1-2 will be described together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that delivery the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it connects to right atrium RA.

Inter-atrial septum IS and fossa ovalis FO are also shown in FIG. 2. Inter-atrial septum IS is the wall that separates right atrium RA from left atrium LA. Fossa ovalis FO is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FO. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FO.

Septal Occluder with Membrane Gates

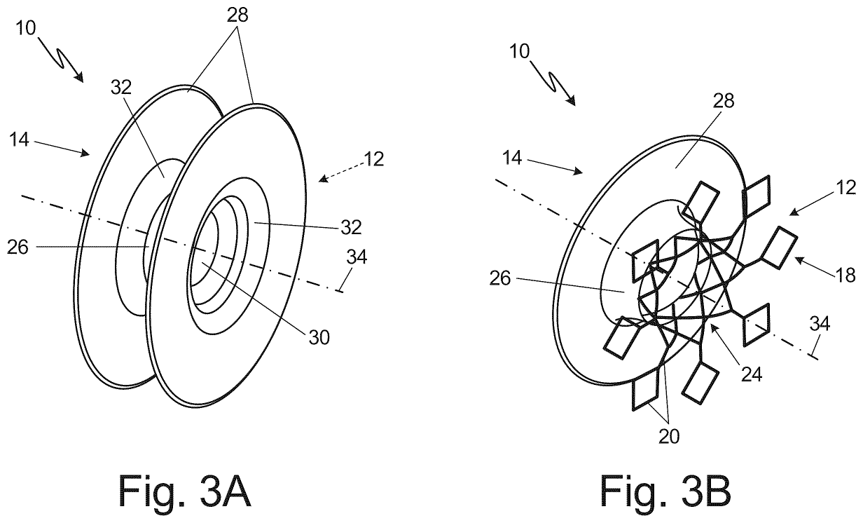

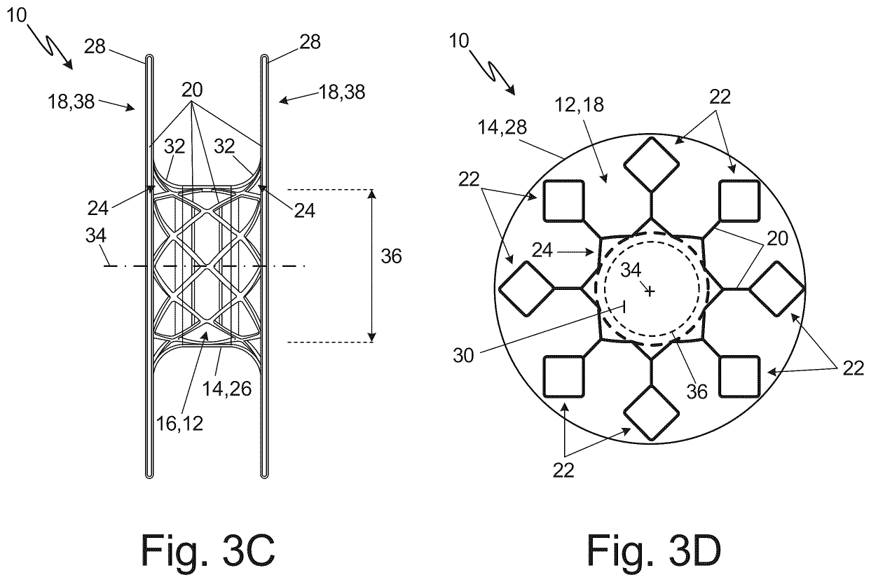

FIG. 3A is an isometric view of an example septal occluder 10 that includes membrane gates. FIG. 3B is an isometric view of septal occluder 10 with a portion of flexible skin 14 removed to show an end section and cylindrical section of structural framework 12. FIG. 3C is a side view of septal occluder 10 with half of flexible skin 14 removed to show structural framework 12 of septal occluder 10. FIG. 3D is an end view of septal occluder 10 with an end face of flexible skin 14 removed to reveal the end section of structural framework 12. FIGS. 3A, 3B, 3C, and 3D are discussed together.

Septal occluder 10 includes structural framework 12 and flexible skin 14. Structural framework 12 includes annular section 16, at least one end section 18, struts 20, and may include pads 22 and/or transition 24. Flexible skin 14 includes annular skin 26, at least one end skin 28, at least one membrane gate 30, and may include transition skin 32. Centerline axis 34 extends through a geometric center of septal occluder 10.

FIGS. 3A, 3B, 3C, and 3D depict the example septal occluder 10 equipped with two membrane gates 30, each membrane gate 30 associated with opposite ends of septal occluder 10. While two membrane gates 30 are shown in this example, other examples of septal occluder 10 can include a single membrane gate 30. In further examples, septal occluder 10 can include more than two membrane gates 30.

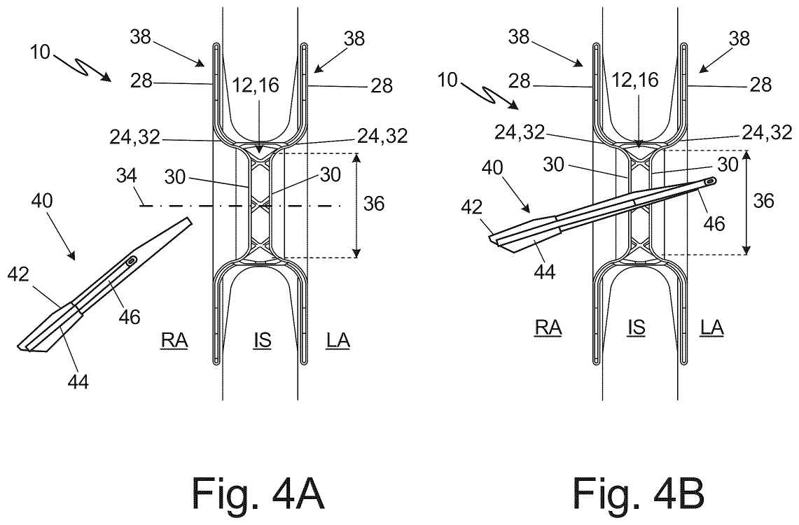

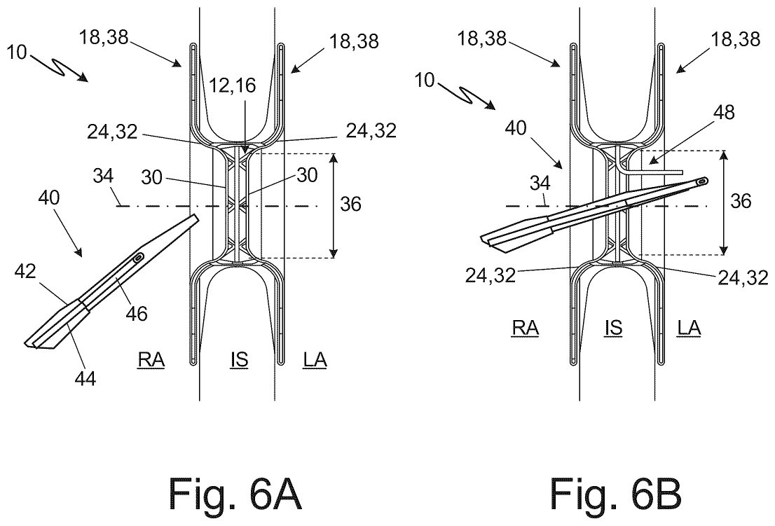

Further, FIGS. 3A, 3B, 3C, 3D depict septal occluder 10 in an undeflected or neutral state. Prior to installation, septal occluder 10 may deflect to conform to an interior surface of a sheath of a catheter, or other delivery device. After installation, septal occluder 10 may conform to topographical features and/or shape of the inter-atrial septum and/or an opening through the inter-atrial septum as shown by FIGS. 4A, 4B, 6A, and 6B.

Annular section 16 extends along and circumscribes centerline axis 34 to define opening 36. Annular section 16 is joined to at least one end section 18 and, in some examples, is interposed between two end sections 18. Each end section 18 extends radially outward relative to centerline axis 34 from annular section 16. In some examples, such as the example depicted in FIGS. 3A-3D, annular section 16 is cylindrical and concentric to centerline axis 34 and opening 36 defines a bore. Structural framework 12 can define transitions 24 from annular section 16 to each end section 18. For instance, each transition 24 can be formed by struts 20 of structural framework 12 that follow a radius or a chamfer between annular section 16 and each end section 18.

Structural framework 12 is formed by struts 20, each strut 20 connected to one or more adjacent struts 20 to form a grid or a mesh. Structural framework 12 defines the shape of septal occluder 10 and supports flexible skin 14 such that flexible skin 14 takes the shape of structural framework 12. The grid (or mesh) of structural framework 12 can be formed by struts 20 arranged into a variety of shapes and patterns, non-exclusive examples of these shapes and patterns are described below.

Struts 20 of annular section 16 may define a grid pattern, with some struts 20 extending parallel to centerline axis 34 and other struts 20 extending in a circumferential direction about centerline axis 34, or extending tangentially to a circumferential direction about centerline axis 34. In other examples, struts 20 may define a helical pattern, with struts 20 extending obliquely to centerline axis 34 as viewed from a radial plane containing centerline axis 34, or a plane offset and parallel to said radial plane. In some examples, struts 20 of annular section 16 can include two or more groups of struts 20, each strut group extending in a circumferential direction (or in a direction tangent to the circumferential direction) about centerline axis 34 to form a helical pattern. In such examples, at least two of the strut groups extend about centerline axis 34 at different helical angles such that at least one group of struts 20 is arranged at an oblique angle or a perpendicular angle to struts 20 of another helical group as shown in FIG. 3C for example.

Struts 20 of end sections 18 may also form a grid pattern, with struts extending along a plane or conic surface defining a shape of end section 18. For instance, struts 20 may include struts 20 extending perpendicularly or obliquely to centerline axis 34 as viewed from an end view normal to centerline axis 34. Some struts 20 within such patterns intersect other struts 20 at perpendicular or oblique angles. In other examples, some struts 20 of end section 18 extend circumferentially (or tangentially to the circumferential direction) about centerline axis 34 while other struts 20 of end section 18 intersect circumferentially extending struts 20 (or struts 20 extending tangentially to a circumferential direction) extending radially and/or extending obliquely to centerline axis 34. In other examples, struts 20 of end sections 18 can define a spiral shape extending about centerline axis 34 with some struts 20 intersecting spirally extending struts 20. In still other examples, such as the example depicted by FIGS. 3B and 3D, struts 20 of end sections 18 can extend radially outward relative to centerline axis 34, each strut 20 cantilevered from annular section 16 and circumferentially spaced from adjacent struts 20 about centerline axis 34. In no example of the present example do struts 20 span opening 36 defined by annular section 16.

Some examples of structural framework 12 include one or more pads 22. Pads 22 are struts 20 formed into a closed shape that is distinct from struts 20 defining a mesh or a grid. Example shapes of pads 22 include struts 20 forming a circle, oval, square, diamond, triangle, rhombus, or other polygonal shape. As depicted by FIG. 3D, pads 22 can be attached to distal ends of cantilevered struts 20.

Flexible skin 14 attaches to structural framework 12. In some examples, flexible skin 14 defines an exterior layer and/or an interior layer of septal occluder 10 with structural framework 12 arranged between the exterior layer and the interior layer. Portions of flexible skin 14 disposed inboard of structural framework 12 relative to centerline axis 34 form an interior layer of septal occluder 10. Inboard portions of flexible skin 14 are disposed between centerline axis 34 and structural framework 12 and can be attached to an interior side of structural framework 12. Portions of flexible skin 14 disposed outboard of structural framework 12 relative to centerline axis 34 form an exterior layer of septal occluder 10. Structural framework 12 is disposed between centerline axis 34 and portions of flexible skin 14 forming the exterior layer, which can be attached to an exterior side of structural framework 12. In other examples, structural framework 12 is partially or fully embedded within flexible skin 14. Flexible skin 14 may enclose structural framework 12 completely such that no strut 20 of structural framework 12 is exposed. In other examples, some struts 20, or portions of struts 20, can be exposed. For example, as best depicted by FIG. 4B, interior-facing portions of structural framework 12 between membrane gates 30 (e.g., portions of annular section 16 facing centerline axis 34) can be exposed or uncovered by flexible skin 14 after membrane gates 30 are pierced.

Annular skin 26 and end skins 28 define portions of flexible skin 14 attached to annular section 16 and end section 18, respectively. Fixation walls 38 are formed by end sections 18 and corresponding end skins 28. Membrane gates 30 extend from end skins 28 to span opening 36 (or bore) defined by annular section 16. In some examples, membrane gates 30 are aligned with an exterior most portion of end skins 28 such that membrane gates 30 are coincident with respective end skins 28. In other examples, membrane gates 30 can be spaced inward along centerline axis 34 from respective end skins 28 to form a depression. The depression mimics the natural depression of the foramen ovale that practitioners use to locate a transseptal puncture site in the inter-atrial septum.

In the example best depicted by FIG. 3D, end skin 28 connects to each cantilevered strut 20 and pad 22 of end section 18. Since the distal ends of cantilevered struts 20 (i.e., radially outer ends relative to centerline axis 34) are not joined to adjacent structs in the circumferential direction, fixation wall 38 of septal occluder 10 can be collapsed inward toward centerline axis 34 more readily to facilitate retention of septal occluder 10 into a sheath of a catheter or other delivery device. As depicted, septal occluder 10 includes eight cantilevered struts 20 for each end section 18. In other examples, end section 18 can include fewer or more than eight cantilevered struts 20. Each end section 18 can include the same number of cantilevered struts 20 in some examples. In other examples, end sections 18 can include different numbers of cantilevered struts 20. Cantilevered struts 20 can be equally spaced circumferentially about centerline axis 34 as shown in FIG. 3D or have variable circumferential spacing about centerline axis 34.

Struts 20 and pads 22 of structural framework 12 and flexible skin 14 are each formed from a resilient material suitable for implantation into a human patient. Acceptable materials are formable into complex shapes, such as the depicted shapes of annular section 16 and end sections 18. For example, struts 20 and pads 22 can be formed from a nickel titanium alloy (e.g., nitinol), which can be formed by a mold and set into a shape with a thermal process. Flexible skin 14 can be formed from expanded polytetrafluoroethylene (ePTFE) or polytetrafluoroethylene (PTFE) among other potential materials.

Annular section 16 and membrane gate 30 allow septal occluder 10 to be punctured after implantation to perform subsequent transseptal procedures. Punctures through septal occluder 10 during transseptal procedures can be closed using a second septal occluder 10 or, alternatively, a conventional septal occluder. As such, septal occluder 10 can remain in place allowing optimal puncture sites to be reused during subsequent transseptal procedures.

FIGS. 4A and 4B are cross-sectional views of the septal occluder taken along centerline axis 34, each figure depicting septal occluder 10 implanted within inter-atrial septum IS of a patient's heart. FIG. 4A depicts septal occluder 10 prior to piercing membrane gates 30. FIG. 4B depicts septal occluder 10 after membrane gates 30 are pierced by needle catheter 40. FIG. 4A and FIG. 4B are discussed together.

As depicted by FIGS. 4A and 4B, fixation walls 38 of septal occluder 10 engage opposite sides of inter-atrial septum IS. Prior to piercing septal occluder 10, membrane gates 30 span opening 36 (or bore) of annular section 16, obstructing a path through the patient's inter-atrial septum IS. As shown by FIG. 4A, septal occluder 10 includes transitions 24 between each of end section 18 and annular section 16. Structural framework 12 follows a curved path between annular section 16 and respective end sections 18 that approximates a radius. Attached to transitions 24, transition skins 32 define a curved path that follows an outside radius between end skins 28 and respective transition skins 32 and follows an inside radius between transition skins 32 and respective membrane gates 30. Septal occluder 10 includes two membrane gates 30, each membrane gate 30 extending from one of transition skins 32 or one of end skins 28.

Membrane gates 30 are spaced inward along centerline axis 34 from respective end skins 28 to form depressions at opposite ends of septal occluder 10. When installed within the inter-atrial septum IS of a patient, transition skins 32 follow a curved path between end skin 28 and membrane skin 30. This surface profile is similar to the surface profile of the fossa ovalis FO in relation the surrounding inter-atrial septum IS, which aids practitioners to locate septal occluder 10 during a transeptal occluder in a similar manner as the practitioner locates the fossa ovalis FO.

Septal occluder 10 can be pierced by needle catheter 40. FIGS. 4A and 4B depict a distal tip of needle catheter 40, which includes sheath 42, inner tube 44, and needle 46. Sheath 42 circumscribes inner tube 44 and needle 46 to protect each during a transseptal procedure. Sheath 42 can include a tapered tip to aid advancement of needle catheter 40 into a patient's heart. Needle 46 is attached to a distal end of inner tube 44. Sheath 42 and inner tube 44 can be independently manipulated by a practitioner from a proximal end of needle catheter 40 using one or more of a handle, a knob, and/or a plunger. Advancing inner tube 44 relative to sheath 42 advances needle 46 distally until needle 46 protrudes from a distal end of sheath 42 to pierce membrane gate 30. Once membrane gate 30 is punctured, needle 46 can be retracted within sheath 42. Further advancement of sheath 42 expands an opening through membrane gate 30. If additional membrane gates 30 exist, the foregoing process repeats for each gate 30 until sheath 42 protrudes through septal occluder 10. Needle 46 may be retracted and removed from catheter 40 in preparation of a subsequent procedure.

After piercing membrane gates 30, as shown by FIG. 4B, a path between the patient's right atrium RA and left atrium LA is defined through opening 36 of septal occluder 10. Membrane gates 30 remain at least partially attached to end skins 28. In some instances, needle catheter 40 pierces membrane gates 30 while the radially outer periphery of membrane gates 30 remains attached to respective end skins 28. In other instances, membrane gate 30 may form a flap formed when part of the radially outer periphery of membrane gates 30 detaches from respective end skins 28. In each instance, subsequent installation of a second septal occluder 10 (or a conventional septal occluder) within opening 36 may prevent detachment of membrane gates 30.

Septal Occluder with Membrane and Mechanical Gates

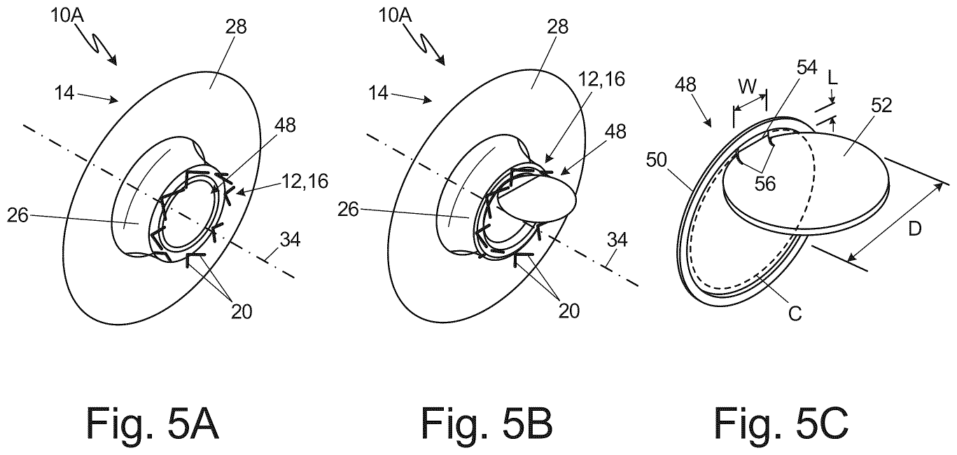

FIGS. 5A and 5B depict another example of septal occluder 10A, which includes mechanical gate 48 in addition to membrane gates 30. Septal occluder 10A has generally the same configuration as septal occluder 10 shown in FIGS. 3A-4B. Components of septal occluder 10A with identical numbering as corresponding components of septal occluder 10 are formed, positioned, and function in the same or similar way as described with respect to septal occluder 10 shown in FIGS. 3A-4B. FIG. 5A and FIG. 5B are isometric cross-sectional views of septal occluder 10A taken along a plane normal to centerline axis 34 and between one of membrane gates 30 and mechanical gate 48. FIG. 5A depicts mechanical gate 48 in a closed position, and FIG. 4B depicts mechanical gate 48 in an open position. Mechanical gate 48 attaches to structural framework 12. In the depicted example, an outer periphery of mechanical gate 48 attaches to annular section 16 of structural framework 12 between membrane gates 30.

FIG. 5C is an isometric view of mechanical gate 48 in the open position. Mechanical gate includes ring 50, flap 52, and hinge member 54. Ring 50 circumscribes flap 52 in the closed position. Hinge member 54 connects ring 50 to flap 52 and permits flap 52 to pivot with respective to ring 50. An outline of flap 52 in the closed position is indicated by dashed lines C in FIG. 5C.

Ring 50 can be attached to structural framework 12 by, for example, sutures threaded through eyelets of ring 50 and around struts 20 of annular section 16. In other examples, ring 50 can be bonded to struts 20 of annular section 16 through a thermal process. In the closed position, an outer periphery of flap 52 is spaced from an inter periphery of ring 50 to form a gap. As depicted in FIGS. 5A, 5B, and 5C, the gap between ring 50 and flap 52 is exaggerated. In some examples, the gap between ring 50 and flap 52 can be reduced such that an inner periphery of ring 50 contacts an outer periphery of flap 52 at one or more locations about the outer periphery of flap 52.

Hinge member 54 has a width W that is a fraction of diameter D of flap 52 to allow flap 52 to pivot with respect to ring 50. In some examples, width W of hinge member 54 is less than or equal to twenty-five percent the diameter D of flap 52. In other examples, width W of hinge member 54 is less than or equal to twenty percent the diameter D of flap 52. In still other examples, width W of hinge member 54 is less than or equal to fifteen percent the diameter D of flap 52. In yet other examples, width W of hinge member 54 can be less than or equal to ten percent the diameter D of flap 52. In each instance, width W of hinge member 54 is at least five percent the diameter D of flap 52. Additionally, flap 52 may include cuts 56 on each side of hinge member 54. Cuts 56 effectively increase length L of hinge member 54.

Mechanical gate 48 is formed from a resilient material suitable for installation into a human patient. Acceptable materials return to an undeflected or neutral state following deflection from an external source (e.g., a force applied by a catheter during a transseptal procedure). For example, ring 50, flap 52, and hinge member 54 of mechanical gate 48 can be formed from a nickel titanium alloy (e.g., nitinol), which can be set into an initial, undeflected position (i.e., the closed position) with a thermal process. Accordingly, mechanical gate 48 can deflect into an open position, such as by a catheter during a transseptal procedure, and return to an undeflected position (i.e., a closed position) once the catheter is removed. Once mechanical gate 48 assumes the closed position, the path through septal occluder 10A is substantially closed without using a second septal occluder as can be used to close septal occluder 10 as described above.

FIGS. 6A and 6B are cross-sectional views of septal occluder 10A taken along centerline axis 34, each figure depicting septal occluder 10A implanted within inter-atrial septum IS of a patient's heart. FIG. 6A depicts septal occluder 10A prior to piercing membrane gates 30 and before mechanical gate 48 is opened. FIG. 6B depicts septal occluder 10A after membrane gates 30 are pierced and mechanical gate 48 is opened by needle catheter 40. FIG. 6A and FIG. 6B are discussed together.

As depicted by FIGS. 6A and 6B, fixation walls 38 of septal occluder 10A engage opposite sides of inter-atrial septum IS. Prior to piercing septal occluder 10A, as shown by FIG. 6A, membrane gates 30 and mechanical gate 48 span opening 36 (or bore) of annular section 16, obstructing the opening through the patient's inter-atrial septum IS. As shown by FIG. 6A, septal occluder 10A includes transitions 24 between each of end section 18 and annular section 16. Structural framework 12 follows a curved path that approximates an outside radius between annular section 16 and respective end sections 18. Attached to transitions 24, transition skins 32 define a curved path that follows the outside radius defined by structural framework 12 and an inside radius between end skins 28 to respective membrane gates 30. Membrane gates 30 are also offset inward from respective end skins 28 along centerline axis 34 to define depressions on each side of septal occluder 10. Mechanical gate 48 attaches to annular section 16 of structural framework 12 and has a position disposed between membrane gates 30 along centerline axis 34. Accordingly, mechanical gate 48 is contained within flexible skin 14 of septal occluder 10A. Membrane gates 30 can be pierced by advancing needle 46 of catheter 40 as described in reference to septal occluder 10 shown in FIGS. 4A-4B. Similarly, mechanical gate 48 can be displaced by advancing needle catheter 40 through mechanical gate 48 with needle 46 retracted within sheath 42 of catheter 40.

After piercing membrane gates 30 and opening mechanical gate 48, as shown by FIG. 6B, a pathway between the patient's right atrium RA and left atrium LA forms through opening 36 of septal occluder 10A. Once membrane gates 30 are pierced, membrane gates 30 remain at least partially attached to end skins 28. In some instances, a needle catheter pierces membrane gates 30 while the radially outer periphery of membrane gates 30 remains attached to respective end skins 28. In other instances, membrane gate 30 may form a flap when part of the radially outer periphery of membrane gates 30 detaches from respective end skins 28. Mechanical gate 48 is biased towards the closed position by selecting a material for mechanical gate 48 that tends to return to a neutral position. For example, mechanical gate 48 can be formed from a nickel titanium alloy (e.g., nitinol). A catheter, or other device, used during the transseptal procedure prevents mechanical gate 48 from closing so long as the catheter, or other device, extends through septal occluder 10A as shown by FIG. 6B. Once the catheter, or other device, is withdrawn from septal occluder 10A, mechanical gate 48 returns to the closed position. Strain imposed on the hinge member 54 by being deflected by the catheter or other device imposes a restoring force on flap 52. The restoring force returns flap 52 to the closed position (i.e., neutral position).

### Delivery Catheter and Septal Occluder

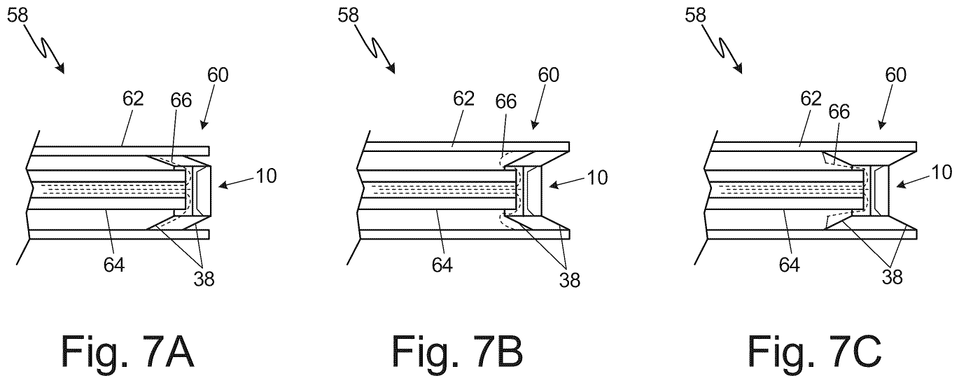

FIGS. 7A, 7B, and 7C are schematic views of system 58 for implanting septal occluder 10 (or occluder 10A). While the following description refers to septal occluder 10, it is understood that any of the following features of system 58 can be implemented using septal occluder 10A, which includes a mechanical gate. As depicted in FIGS. 7A, 7B, and 7C, system 58 includes delivery catheter 60 and septal occluder 10, which is retained at a distal tip of catheter 60. Catheter 60 includes sheath 62 and inner tube 64, which can be independently manipulated by a practitioner from a proximal end (not shown) of catheter 60 outside the patient's body. In each configuration, fixation walls 38 of septal occluder 10 are folded inward towards centerline axis 34 of septal occluder 10. In some examples, annular section 16 compresses inward towards centerline axis 34 to reduce a diameter of opening 36 as facilitated by the grid or mesh pattern of structural framework 12. FIG. 7A and FIG. 7B depict fixation walls 38 folded in the same direction. In FIG. 7A, fixation walls 38 fold toward the proximal end of catheter 60. In FIG. 7B, fixation walls 38 fold toward the distal end of catheter 60. Fixation walls 38 can be folded in opposite directions as shown in FIG. 7C. In this example, a proximal fixation wall 38 folds towards the proximal end of catheter 60 and a distal fixation wall 38 folds towards the distal end of the catheter 60. The proximal fixation wall 38 is located on an end of septal occluder 10 (or occluder 10A) that faces the proximal end of catheter 60, and the distal fixation wall 38 is located on an opposite end of septal occluder 10 (or occluder 10A) that faces the distal end of catheter 60.

Septal occluder 10 can be retained frictionally within catheter 60. When structural framework 12 of septal occluder 10 is constructed from a shape memory alloy (e.g., a nickel titanium alloy (nitinol)), structural framework 12 and, hence, the septal occluder returns to a neutral or undeflected shape. Installation of septal occluder 10 into catheter 60 deflects fixation walls 38 towards the proximal or distal ends of catheter 60. Strain imposed on structural framework 12 produces a frame restoring force, tending to return fixation walls 38 to the undeflected shape. When installed within catheter 60, the frame restoring force produced by structural framework 12 reacts against an interior surface of sheath 62. Sheath 62 retains septal occluder in a direction normal to catheter 60, and the frame restoring force retains septal occluder 10 along a longitudinal direction of catheter 60.

In some examples, septal occluder 10 can be retained to or within catheter 60 by wire 66, which are represented in FIGS. 7A, 7B, and 7C by dashed lines. In such examples, wire 66 extends from a proximal end of catheter 60 through an interior of sheath 62 and/or an interior of inner tube 64 to septal occluder 10. Septal occluder 10 can include eyelets distributed about a radial outer periphery of end sections 18 (shown in FIGS. 3A-3D). Wire 66 extends from within sheath 62 and/or inner tube 64, through each eyelet of each end section 18, and returns to the proximal end of catheter 60 through sheath 62 and/or inner tube 64. Wire 66 can be disconnected from septal occluder 10 (and occluder 10A) by breaking wire 66. Alternatively, withdrawing wire 66 from the proximal end of catheter 60 withdraws wire 66 through eyelets of end sections 18 through sheath 62 and/or inner tube 64 to disconnect septal occluder 10 from delivery catheter 60.

### Septal Occluder Implant Procedure

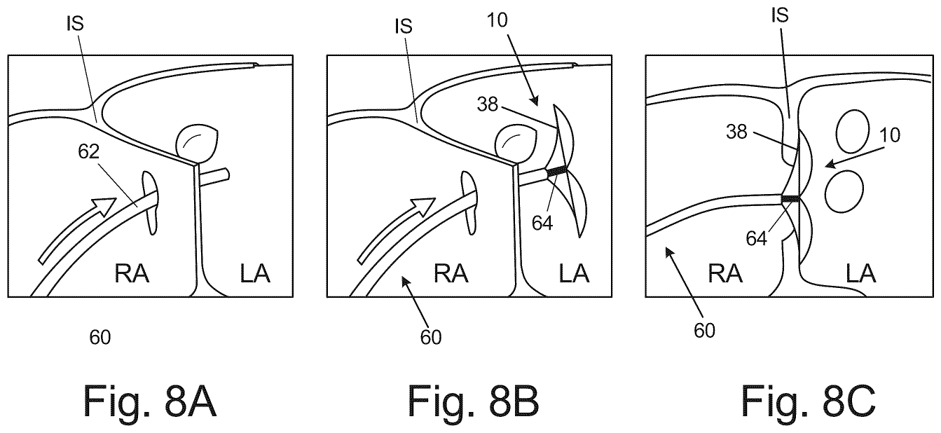

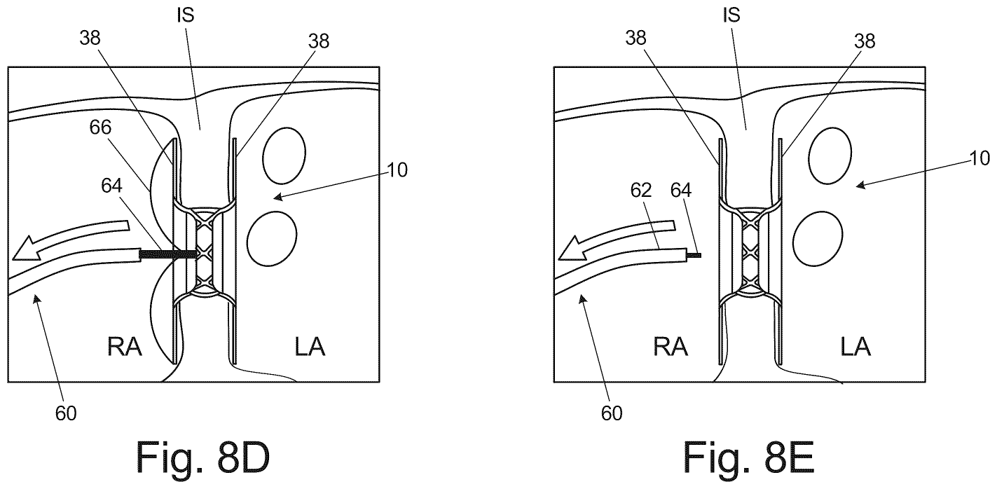

FIGS. 8A, 8B, 8C, 8D, and 8E are schematic views that illustrate an implantation sequence of septal occluder 10 (or occluder 10A) into a patient's heart using catheter 60. While the following sequence describes implanting septal occluder 10, it is understood that the following sequence is equally applicable to septal occluder 10A that includes a mechanical gate. FIG. 8A depicts catheter 60 inserted through an opening in a patient's inter-atrial septum IS from right atrium RA of the patient's heart. A distal tip of catheter 60 retains septal occluder 10 as described by FIGS. 7A, 7B, and 7C. In FIG. 8B, sheath 62 is withdrawn relative to inner tube 64 to expose a distal fixation wall 38 of septal occluder 10. After released from sheath 62, distal fixation wall 38 expands outward, returning to an undeflected shape. Withdrawing sheath 62 and inner tube 64 concurrently draws distal fixation wall 38 towards the patient's inter-atrial septum IS until the distal fixation wall 38 abuts the inter-atrial septum IS as shown in FIG. 8C. FIG. 8D depicts releasing proximal fixation wall 38 by withdrawing sheath 62 with respect to inner tube 64. After released from sheath 62, proximal fixation wall 38 expands outward towards an undeflected shape. In this position, proximal fixation wall 38 abuts inter-atrial septum IS. Catheter 60 is withdrawn in FIG. 8E. If wires 66 are used to retain septal occluder 10 within catheter 60, wires 66 are withdrawn through sheath 62 and/or inner tube 64 from a proximal end of catheter 60. Alternatively, wires 66 may be broken by withdrawing catheter 60 with respect to septal occluder 10 after septal occluder 10 is fully deployed and implanted within inter-atrial septum IS.

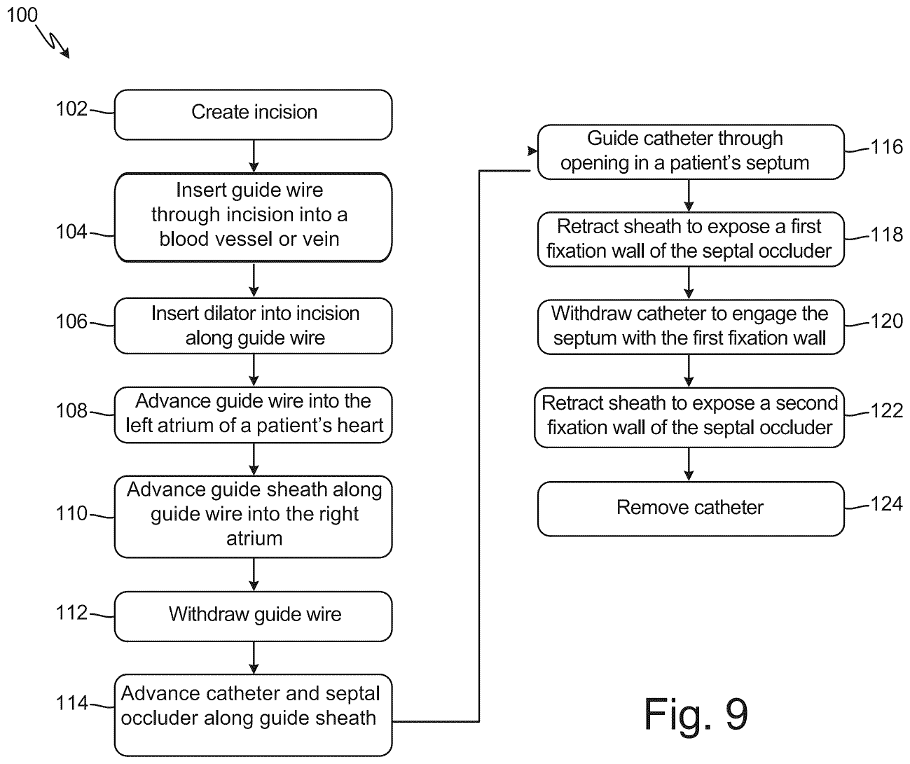

FIG. 9 is a flowchart describing method 100 of implanting a septal occluder in a patient's heart. Method 100 includes steps 102, 104, 106, 108, 110, 112, 114, 116, 118, 120, 122, and 124. The sequence depicted is for illustrative purposes only and is not meant to limit the method 100 in any way, as it is understood that the portions of the method can proceed in a different logical order, additional or intervening portions can be included, described portions of the method can be divided into multiple portions, or described portions of the method can be omitted without detracting from the purpose as described below.

Method 100 begins by preparing an incision site for delivery catheter 60. Step 102 includes creating an incision at a location on the patient's body that provides access to a blood vessel. For example, the femoral vein can be accessed through an incision within a region of the patient's groin. In step 104, a practitioner inserts a guide wire through the incision to pierce the patient's blood vessel (e.g., a femoral vein). The practitioner introduces a dilator into the incision along the guide wire to enlarge the incision site in step 106. An introducer may be installed into the incision site in step 106 to further prepare the incision site for delivery catheter 60.

After preparation of the incision site, method 100 involves gaining access to the patient's right atrium for delivery catheter 60. In step 108, the practitioner advances the guide wire from the incision site through the blood vessel(s) into the right atrium of the patient's heart. For example, this may be accomplished by advancing the guide wire though the patient's femoral vein and inferior vena cava IVC into the right atrium RA of the patient's heart. In step 110, a guide sheath is inserted onto the guide wire and advanced along the guide wire into the patient's right atrium. After guide sheath is within the patient's right atrium, guide wire can be withdrawn from the patient in step 112. In step 114, catheter 60 is advanced along the guide sheath into the patient's right atrium.

Practitioners deploy septal occluder 10 (or septal occluder 10A) within an opening in the inter-atrial septum IS by manipulating sheath 62 and inner tube 64 from a proximal end of catheter 60. The proximal end of catheter 60 may include a handle comprising an assembly of knobs or plungers used to manipulate sheath 62 independently or concurrently with inner tube 64. In step 116, practitioners guide catheter 60 through the opening in the inter-atrial septum IS. As the practitioner advances catheter 60 through the inter-atrial septum IS, septal occluder 10 (or occluder 10A) remains collapsed within sheath 62, conforming to an inner surface of catheter 60. After the distal tip of catheter 60 advances into the patient's left atrium, the practitioner withdraws sheath 62 relative to inner tube 64 to expose a distal fixation wall 38 of septal occluder 10 (or occluder 10A) in step 118. When the distal fixation wall 38 is exposed from sheath 62, it will expand outwards. Withdrawing sheath 62 can include translating sheath 62 towards a proximal end of catheter 60 by, for example, manipulating a handle, plunger, or knob at the proximal end of catheter 60 that translates a distal tip of sheath 62 towards the patient's right atrium while maintaining a position of inner tube 64. Alternatively, a practitioner may advance inner tube 64 relative to sheath 62 while maintaining or withdrawing sheath 62. In this instance, inner tube 64 displaces septal occluder 10 (or occluder 10A) relative to sheath 62 to expose a distal fixation wall 38.

In step 120, the practitioner withdraws catheter 60, maintaining relative positions of sheath 62 and inner tube 64, to draw distal fixation wall 38 into contact with the patient's inter-atrial septum IS. While holding the distal fixation wall 38 against the inter-atrial septum IS within the left atrium, the practitioner exposes a proximal fixation wall 38 of septal occluder 10 (or occluder 10A) in step 122. The practitioner exposes the proximal fixation wall 38 of septal occluder 10 (or occluder 10A) by withdrawing sheath 62 relative to inner tube 64 while maintaining a position of inner tube 64. When the proximal fixation wall 38 is exposed from sheath 62, it will expand outwards and abut inter-atrial septum IS in the right atrium. In step 124, the practitioner withdraws catheter 60 from the patient's body through the incision site and closes the incision site using techniques known in the art. Withdrawing catheter 60 from the patient can include withdrawing or breaking wire 66 or wires 66 attaching septal occluder 10 (or occluder 10A) to catheter 60.

### Septal Occluder Recrossing Procedure

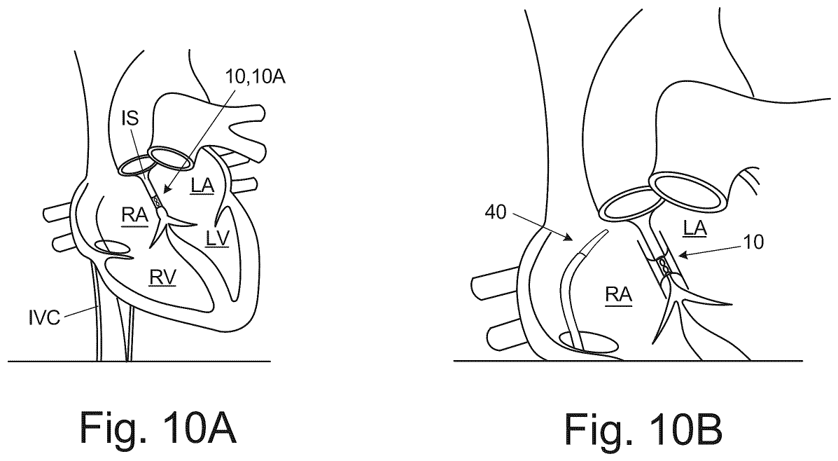

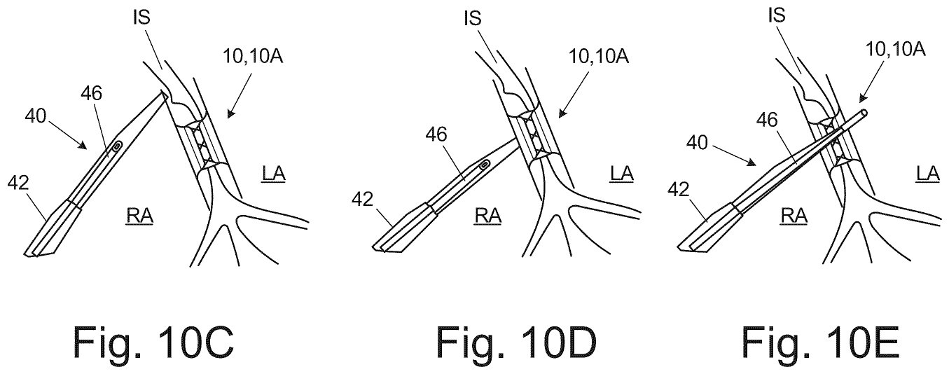

FIGS. 10A, 10B, 10C, 10D, and 10E are schematic views that describe a process for recrossing septal occluder 10 (or septal occluder 10A). Septal occluder 10 can be pierced by needle catheter 40, a distal tip of which is depicted in FIGS. 4A, 4B, 6A, and 6B. FIG. 10A illustrates a guide wire advanced into a patient's right atrium RA via inferior vena cava IVC. A practitioner inserts needle catheter 40 onto guide wire through an introducer at the incision site. FIG. 10B depicts advancing needle catheter 40 along the guide wire through inferior vena cava IVC and into right atrium RA of a patient's heart. As represented by FIG. 10C, the practitioner manipulates needle catheter 40 along inter-atrial septum IS until the distal tip of needle catheter 40 rests on or adjacent to the limbus fossa ovalis. In FIG. 10D, the practitioner manipulates needle catheter 40 to engage septal occluder 10 (or occluder 10A). Septal occluders equipped with a depression aid this step by mimicking the topographical features of the fossa ovalis. FIG. 10E depicts the practitioner advancing needle 46 to protrude from sheath 42. As depicted, needle 46 pierces membrane gates 30 of septal occluder 10 (or occluder 10A).

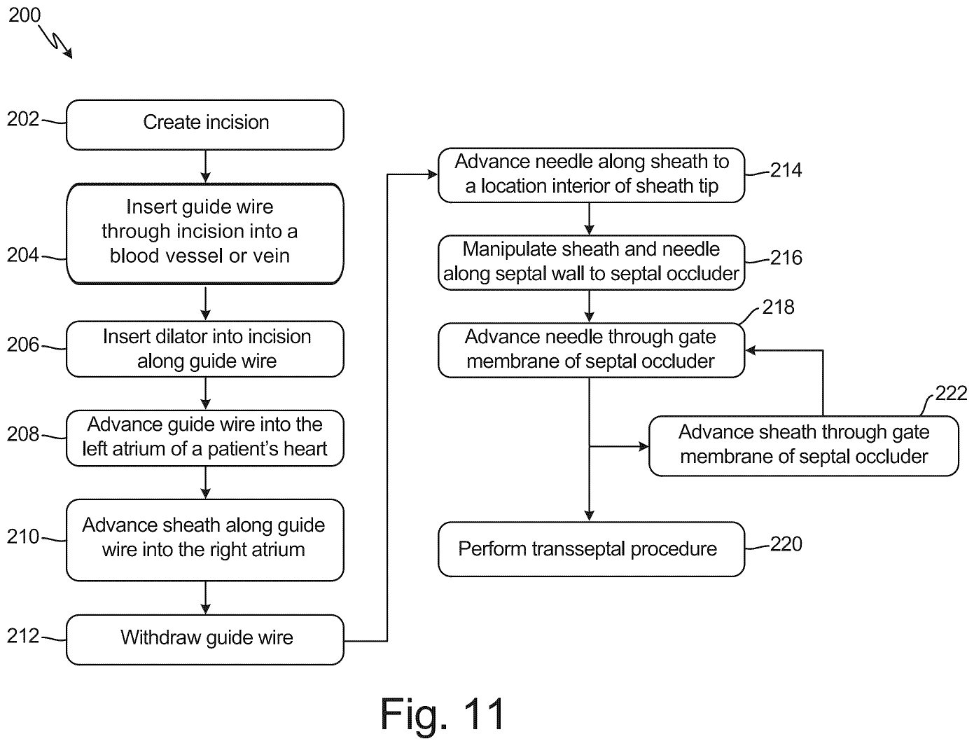

FIG. 11 is a flowchart describing method 200 for recrossing septal occluder 10 equipped with membrane gates 30 or for recrossing septal occluder 10A equipped with membrane gates 30 and mechanical gate 48. Method 200 includes steps 202, 204, 206, 208, 210, 212, 214, 216, 218, 220, and 222. The sequence depicted is for illustrative purposes only and is not meant to limit the method 100 in any way as it is understood that the portions of the method can proceed in a different logical order, additional or intervening portions can be included, or described portions of the method can be divided into multiple portions, or described portions of the method can be omitted without detracting from the purpose as described below.

Method 200 begins by preparing an incision site for needle catheter 40. Step 202 includes creating an incision at a location on the patient's body that provides access to a blood vessel. For example, the femoral vein can be accessed through an incision within a region of the patient's groin. In step 204, a practitioner inserts a guide wire through the incision to pierce the patient's blood vessel (e.g., a femoral vein). The practitioner introduces a dilator into the incision along the guide wire to enlarge the incision site in step 206. An introducer may be installed into the incision site in step 206 to further prepare the incision site for needle catheter 40.

After preparation of the incision site, method 200 involves gaining access to the patient's right atrium for needle catheter 40. In step 208, the practitioner advances the guide wire from the incision site through the blood vessel(s) into the right atrium of the patient's heart. For example, this may be accomplished by advancing the guide wire though the patient's femoral vein and inferior vena cava into the right atrium of the patient's heart. In step 210, needle catheter 40 is inserted onto the guide wire and advanced along the guide wire into the patient's right atrium by the practitioner. After catheter 40 is within the patient's right atrium, guide wire can be withdrawn from the patient in step 212.

If not already contained within needle catheter 40, the practitioner advances needle 46 into and through sheath 42 of needle catheter 40 to a location within a distal tip of catheter 40 in step 214. In step 216, the practitioner manipulates needle catheter to guide its distal tip along the inter-atrial septum IS to septal occluder 10. Step 116 can include guiding the distal tip of needle catheter 40 along the inter-atrial septum IS until the limbus fossa ovalis. The limbus fossa ovalis is a prominent feature or ridge that defines a perimeter of the fossa ovalis. Step 216 may also include the practitioner identifying septal occluder 10 (or occluder 10A) by its depression. The depression of the septal occluder 10 is defined by a membrane gate that is offset inward relative to a fixation wall of the septal occluder. Septal occluders with a depression features mimic the topographical features of the fossa ovalis.

In step 218, the practitioner advances needle 46 distally relative to sheath 42 until needle 46 protrudes from sheath 42 and pierces membrane gate 30 of septal occluder 10 or septal occluder 10A. If the septal occluder includes a single membrane gate and no mechanical gate, the practitioner now performs steps of a transseptal procedure in step 220. In some instances, the septal occluder has two or more membrane gates and no mechanical gate. In these instances, the practitioner repeats step 218 for each additional membrane gate before executing the transseptal procedure in step 220.

Where septal occluder includes multiple membrane gates and a mechanical gate, such as septal occluder 10A, the practitioner pierces the first membrane gate in step 218. Next, the practitioner withdraws needle 46 within sheath 42 before advancing sheath 42 through mechanical gate 48 in step 222. Subsequently, the practitioner repeats step 218, advancing needle 46 to pierce second membrane gate 30. Once both membrane gates 30 and mechanical gate 48 are opened, the practitioner may perform the transseptal procedure in step 220. As part of performing the transseptal procedure, the practitioner may install an additional septal occluder within the existing septal occluder to close the opening produced during the recrossing procedure. In this case, the septal occluder can be of a design consistent with septal occluder 10 or septal occluder 10A, but also may be a conventional septal occluder that cannot be recrossed during subsequent procedures. Where the septal occluder includes mechanical gate 48, a second septal occluder is not required. Once the catheter or other devices are retracted from septal occluder 10A, mechanical gate 48 returns to a closed position automatically. In the closed position, mechanical gate 48 substantially occludes the bore of septal occluder 10A. Tissue growth may completely close the bore after a recovery period following the transeptal procedure.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein can be performed on a living animal or on a non-living simulation, such as on a cadaver, cadaver heart, anthropomorphic ghost, simulator (e.g., with the body parts, tissue, etc. being simulated), etc.

### Nonexhaustive Discussion of Detailed Examples

The following are non-exclusive descriptions of possible examples of the present invention.

Septal Occluder with Gate

A septal occluder in accordance with an example of this disclosure includes a flexible skin attached to a structural framework. The structural framework includes a plurality of struts interconnected to form a mesh and includes a first end section, a second end section, and a cylindrical section interposed between the first end section and the second end section. The cylindrical section extends along a centerline axis to define a bore. The first end section and the second end section extend radially outward relative to the centerline axis from the cylindrical section. The flexible skin includes a first membrane gate spanning the bore.

The septal occluder of the preceding paragraph can optionally include, additionally and/or alternatively, any one or more of the following features, configurations and/or additional components.

A further example of the foregoing septal occluder, wherein the flexible skin can attach to an interior side and an exterior side of the structural framework relative to the centerline axis to enclose the structural framework.

A further example of any of the foregoing septal occluders, wherein the flexible skin can include a first end skin portion attached to the first end section to define a first fixation wall.

A further example of any of the foregoing septal occluders, wherein the first end skin portion can be spaced along the centerline axis from the first membrane gate to define a first depression.

A further example of any of the foregoing septal occluders, wherein the structural framework further can include a first transition connecting the first end section to the cylindrical section.

A further example of any of the foregoing septal occluders, wherein the first transition follows a first curved path from the first end section to the cylindrical section.

A further example of any of the foregoing septal occluders, wherein the first end section further can include a first plurality of struts.

A further example of any of the foregoing septal occluders, wherein the second end section further can include a second plurality of struts.

A further example of any of the foregoing septal occluders, wherein the first plurality of struts and the second plurality of struts can cantilever in a radial direction from the cylindrical section of the structural framework.

A further example of any of the foregoing septal occluders, wherein the flexible skin can circumferentially connect struts of the first plurality of struts.

A further example of any of the foregoing septal occluders, wherein the flexible skin can circumferentially connect struts of the second plurality struts.

A further example of any of the foregoing septal occluders, wherein the first end section further can include a first plurality of pads.

A further example of any of the foregoing septal occluders, wherein the second end section can include a second plurality of pads.

A further example of any of the foregoing septal occluders, wherein pads of the first plurality of pads can be disposed at respective distal ends of the first plurality of struts.

A further example of any of the foregoing septal occluders, wherein pads of the second plurality of pads can be disposed at respective distal ends of the second plurality of struts.

A further example of any of the foregoing septal occluders, wherein each pad of the first plurality of pads can be formed by struts forming a closed shape.

A further example of any of the foregoing septal occluders, wherein each pad of the second plurality of pads can be formed by struts forming a closed shape.

A further example of any of the foregoing septal occluders, wherein the septal occluder can be sterilized.

A further example of any of the foregoing septal occluders, wherein the flexible skin can include a second membrane gate spanning the bore from the structural framework.

A further example of any of the foregoing septal occluders, wherein the second membrane gate can be spaced from the first membrane gate along the centerline axis.

A further example of any of the foregoing septal occluders, wherein the flexible skin can include a first end skin portion attached to the first end section to define a first fixation wall.

A further example of any of the foregoing septal occluders, wherein the first membrane gate can be spaced from the first end skin portion along the centerline axis to define a first depression.

A further example of any of the foregoing septal occluders, wherein the flexible skin can include a second end skin portion attached to the second end section to define a second fixation wall.

A further example of any of the foregoing septal occluders, wherein the second membrane gate can be spaced from the second end skin portion along the centerline axis to define a second depression.

A further example of any of the foregoing septal occluders, wherein the first membrane gate can be closer to the first end section than the second end section.

A further example of any of the foregoing septal occluders, wherein the second membrane gate can be closer to the second end section than the first end section.

A further example of any of the foregoing septal occluders, wherein the structural framework further can include a first transition connecting the first end section to the cylindrical section.

A further example of any of the foregoing septal occluders, wherein the first transition follows a first curved path from the first end section to the cylindrical section.

A further example of any of the foregoing septal occluders, wherein the structural framework further can include a second transition connecting the second end section to the cylindrical section.

A further example of any of the foregoing septal occluders, wherein the second transition follows a second curved path from the second end section to the cylindrical section.

A further example of any of the foregoing septal occluders can include a mechanical gate attached to the structural framework and spanning the bore.

A further example of any of the foregoing septal occluders, wherein the mechanical gate can include a ring circumscribing the centerline axis, a flap, and a hinge member connecting the ring to the flap.

A further example of any of the foregoing septal occluders, wherein the flap can be displaceable relative to the ring about the hinge member.

A further example of any of the foregoing septal occluders, wherein the hinge member can attach to a radially inner periphery of the ring relative to the centerline axis and a radially outer periphery of the flap relative to the centerline axis.

A further example of any of the foregoing septal occluders, wherein a width of the hinge member measured within a plane of the ring can be greater than or equal to five percent and less than or equal to twenty five percent of a diameter of the flap.

A further example of any of the foregoing septal occluders, wherein the flexible skin can include a second membrane gate spanning the bore from the structural framework.

A further example of any of the foregoing septal occluders, wherein the mechanical gate can be disposed between the first membrane gate and the second membrane gate.

### System Incorporating a Septal Occluder

A system according to an example of this disclosure, among other possible things, includes a catheter and a septal occluder. The catheter includes a handle, a tube, and a sheath. The tube attaches to the handle and the sheath circumscribes at least a portion of the tube. The septal occluder is retained within the sheath and includes a flexible skin attached to a structural framework. The structural framework includes a plurality of struts interconnected to form a mesh. The structural framework includes a first end section, a second end section, and a cylindrical section interposed between the first end section and the second end section. The cylindrical section extends along a centerline axis to define a bore. The first end section and the second end section extend radially outward relative to the centerline axis from the cylindrical section. The flexible skin includes a first membrane gate spanning the bore.

The system of the preceding paragraph can optionally include, additionally and/or alternatively, any one or more of the following features, configurations and/or additional components.

A further example of the foregoing system, wherein the septal occluder can include a first end skin portion attached to the first end section to define a first fixation wall.

A further example of any of the foregoing systems, wherein the septal occluder can include a second end skin portion attached to the second end section to define a second fixation wall.

A further example of any of the foregoing systems, wherein the first membrane gate can be spaced from the first end skin portion along the centerline axis to define a first depression.

A further example of any of the foregoing systems, wherein the second membrane gate can be spaced from the second end skin portion along the centerline axis to define a second depression.

A further example of any of the foregoing systems, wherein the flexible skin can attach to an interior side and an exterior side of the structural framework relative to the centerline axis to enclose the structural framework.

A further example of any of the foregoing systems, wherein the structural framework can include a first transition connecting the first end section to the cylindrical section.

A further example of any of the foregoing systems, wherein the first transition follows a first curved path from the first end section to the cylindrical section.

A further example of any of the foregoing systems, wherein the structural framework can include a second transition connecting the second end section to the cylindrical section.

A further example of any of the foregoing systems, wherein the second transition follows a second curved path from the second end section to the cylindrical section.

A further example of any of the foregoing systems, wherein the first fixation wall and the second fixation wall can be deflected toward a proximal end of the catheter by the sheath.

A further example of any of the foregoing systems, wherein the first fixation wall and the second fixation wall can be deflected toward a distal end of the catheter by the sheath.

A further example of any of the foregoing systems, wherein the first fixation wall can be deflected towards a distal end of the catheter by the sheath.

A further example of any of the foregoing systems, wherein the second fixation wall can be deflected toward a proximal end of the catheter by the sheath.

A further example of any of the foregoing systems, wherein the first end section further can include a first plurality of struts.

A further example of any of the foregoing systems, wherein the second end section further can include a second plurality of struts.

A further example of any of the foregoing systems, wherein the first plurality of struts and can cantilever in a radial direction from the cylindrical section of the structural framework.

A further example of any of the foregoing systems, wherein the second plurality of struts can cantilever in a radial direction from the cylindrical section of the structural framework.

A further example of any of the foregoing systems, wherein the flexible skin can circumferentially connect struts of the first plurality of struts.

A further example of any of the foregoing systems, wherein the flexible skin can circumferentially connect struts of the second plurality struts.

A further example of any of the foregoing systems, wherein the first end section can include a first plurality of pads.

A further example of any of the foregoing systems, wherein the second end section can include a second plurality of pads.

A further example of any of the foregoing systems, wherein pads of the first plurality of pads can be disposed at respective distal ends of the first plurality of struts.

A further example of any of the foregoing systems, wherein pads of the second plurality of pads can be disposed at respective distal ends of the second plurality of struts.

A further example of any of the foregoing systems, wherein each pad of the first plurality of pads can be formed by struts forming a closed shape.

A further example of any of the foregoing systems, wherein each pad of the second plurality of pads can be formed by struts forming a closed shape.

A further example of any of the foregoing systems, wherein the catheter and the septal occluder can be sterilized.

A further example of any of the foregoing systems, wherein the flexible skin can include a second membrane gate spanning the bore from the structural framework.

A further example of any of the foregoing systems, wherein the second membrane gate can be spaced from the first membrane gate along the centerline axis.

A further example of any of the foregoing systems, wherein the first membrane gate can be closer to the first end section than the second end section.

A further example of any of the foregoing systems, wherein the second membrane gate can be closer to the second end section than the first end section.

A further example of any of the foregoing systems, wherein the septal occluder can include a mechanical gate attached to the structural framework and spanning the bore.

A further example of any of the foregoing systems, wherein the mechanical gate can include a ring circumscribing the centerline axis, a flap, and a hinge member connecting the ring to the flap.

A further example of any of the foregoing systems, wherein the flap can be displaceable relative to the ring about the hinge member.

A further example of any of the foregoing systems, wherein the mechanical gate can be disposed between the first membrane gate and the second membrane gate.

A further example of any of the foregoing systems, wherein the hinge member can attach to a radially inner periphery of the ring relative to the centerline axis and a radially outer periphery of the flap relative to the centerline axis.

A further example of any of the foregoing systems, wherein a width of the hinge member measured within a plane of the ring can be greater than or equal to five percent and less than or equal to twenty five percent of a diameter of the flap.

The above method(s) can be performed on a living animal or on a simulation, such as on a cadaver, cadaver heart, anthropomorphic ghost, simulator (e.g., with body parts, heart, tissue, etc. being simulated).

Method for Implanting a Septal Occluder with a Gate

A method of implanting a septal occluder according to an example of this disclosure includes, among other steps, guiding a catheter through an opening in an inter-atrial septum separating a right atrium and a left atrium of a patient's heart. A septal occluder is retained within a distal end the catheter and includes a flexible skin attached to a structural framework. The structural framework includes a plurality of struts interconnected to form a mesh and defines a bore. The flexible skin includes a first membrane gate spanning the bore. The structural framework and the flexible skin form a first fixation wall and a second fixation wall. The method further includes retracting a sheath of the catheter to expose the first fixation wall of the septal occluder and withdrawing the catheter until the first fixation wall abuts the septum. After retracting the sheath to expose the first fixation wall, the first fixation wall expands outward. The method further includes retracting the sheath to expose the second fixation wall after the first fixation wall abuts the inter-atrial septum and removing the catheter while the septal occluder remains engaged to the inter-atrial septum. After retracting the sheath to expose the second fixation wall, the second fixation wall expands outward.

The method of the preceding paragraph can optionally include, additionally and/or alternatively, any one or more of the following features, configurations, additional components, and/or steps.

A further example of the foregoing method can include breaking or withdrawing a plurality of wires connecting the septal occluder to the catheter that disconnects the septal occluder from the catheter.

A further example of any of the foregoing methods, wherein the sheath can deform the first fixation wall and the second fixation wall to conform the septal occluder to an interior surface of the sheath.

A further example of any of the foregoing methods, wherein deformation of the structural framework can cause the structural framework to impose a restoring force on to the sheath that thereby retains the septal occluder to the catheter.

A further example of any of the foregoing methods can include guiding the catheter into the right atrium of the patient.

Method for Recrossing a Septal Occluder with a Gate

A method for recrossing a septal occluder according to an example of this disclosure includes, among other steps, guiding a catheter into a right atrium of a patient's heart. The septal occluder includes a flexible skin attached to a structural framework. The structural framework defines a bore. The flexible skin includes a first membrane gate that spans the bore. The method further includes extending a needle from within the catheter to pierce the first membrane gate of the septal occluder.

The method of the preceding paragraph can optionally include, additionally and/or alternatively, any one or more of the following features, configurations, additional components, and/or steps.

A further example of the foregoing method can include extending the needle from within the catheter to pierce a second membrane gate of the septal occluder.

A further example of any of the foregoing methods can include retracting the needle within the catheter after piercing the first membrane gate.

A further example of any of the foregoing methods can include extending the catheter with the needle retracted within the catheter to deflect a flap of a mechanical gate into an open position.

A further example of any of the foregoing methods can include extending the needle from within the catheter to pierce a second membrane gate after deflecting the flap of the mechanical gate into the open position.

A further example of any of the foregoing methods retracting the catheter through the mechanical gate whereby a restoring force imposed on the mechanical gate returns the flap to a closed position.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A septal occluder comprising:
a structural framework comprising a plurality of struts interconnected to form a mesh, wherein the structural framework comprises:
a first end section;
a second end section; and
a cylindrical section interposed between the first end section and the second end section, wherein the cylindrical section extends along a centerline axis to define a bore, and wherein the first end section and the second end section extend radially outward relative to the centerline axis from the cylindrical section; and
a flexible skin attached to the structural framework, wherein the flexible skin comprises a first membrane gate spanning the bore uninterrupted.

2. The septal occluder of claim 1, wherein the flexible skin attaches to an interior side and an exterior side of the structural framework relative to the centerline axis to enclose the structural framework.

3. The septal occluder of claim 1, wherein the flexible skin further comprises a first end skin portion attached to the first end section to define a first fixation wall.

4. The septal occluder of claim 3, wherein the first end skin portion is spaced along the centerline axis from the first membrane gate to define a first depression.

5. The septal occluder of claim 4, wherein the structural framework further comprises a first transition connecting the first end section to the cylindrical section, wherein the first transition follows a first curved path from the first end section to the cylindrical section.

6. The septal occluder of claim 1,
wherein the first end section further comprises a first plurality of struts and the second end section further comprises a second plurality of struts;
wherein the first plurality of struts and the second plurality of struts cantilever in a radial direction from the cylindrical section of the structural framework; and
wherein the flexible skin circumferentially connects struts of the first plurality of struts and circumferentially connects struts of the second plurality struts.

7. The septal occluder of claim 6,
wherein the first end section further comprises a first plurality of pads and the second end section further comprises a second plurality of pads;
wherein pads of the first plurality of pads are disposed at respective distal ends of the first plurality of struts;
wherein pads of the second plurality of pads are disposed at respective distal ends of the second plurality of struts; and
wherein each pad of the first plurality of pads and each pad of the second plurality of pads are formed by struts forming a closed shape.

8. The septal occluder of claim 1, wherein the septal occluder is sterilized.

9. The septal occluder of claim 1, wherein the flexible skin includes a second membrane gate spanning the bore from the structural framework, and wherein the second membrane gate is spaced from the first membrane gate along the centerline axis.

10. The septal occluder of claim 9, wherein the flexible skin comprises:
a first end skin portion attached to the first end section to define a first fixation wall, wherein the first membrane gate is spaced from the first end skin along the centerline axis to define a first depression; and
a second end skin portion attached to the second end section to define a second fixation wall, wherein the second membrane gate is spaced from the second end skin along the centerline axis to define a second depression.

11. The septal occluder of claim 10, wherein the first membrane gate is closer to the first end section than the second end section, and wherein the second membrane gate is closer to the second end section than the first end section.

12. The septal occluder of claim 11,
wherein the structural framework further comprises a first transition connecting the first end section to the cylindrical section, wherein the first transition follows a first curved path from the first end section to the cylindrical section; and
wherein the structural framework further comprises a second transition connecting the second end section to the cylindrical section, wherein the second transition follows a second curved path from the second end section to the cylindrical section.

13. A septal occluder comprising:
a structural framework comprising a plurality of struts interconnected to form a mesh, wherein the structural framework comprises:
a first end section;
a second end section; and
a cylindrical section interposed between the first end section and the second end section, wherein the cylindrical section extends along a centerline axis to define a bore, and wherein the first end section and the second end section extend radially outward relative to the centerline axis from the cylindrical section;
a flexible skin attached to the structural framework, wherein the flexible skin comprises a first membrane gate spanning the bore; and
a mechanical gate attached to the structural framework that is resiliently displaceable between a closed position spanning the bore and an open position in which the bore is at least partially unobstructed by the mechanical gate.

14. The septal occluder of claim 13, wherein the mechanical gate comprises:
a ring circumscribing the centerline axis;
a flap; and
a hinge member connecting the ring to the flap, wherein the flap is displaceable relative to the ring about the hinge member.

15. The septal occluder of claim 14, wherein the hinge member attaches to a radially inner periphery of the ring relative to the centerline axis and a radially outer periphery of the flap relative to the centerline axis.

16. The septal occluder of claim 13, wherein the flexible skin includes a second membrane gate spanning the bore from the structural framework.

17. The septal occluder of claim 16, wherein the mechanical gate is disposed between the first membrane gate and the second membrane gate.

18. A system comprising:
a catheter comprising:
a handle;
a tube attached to the handle; and
a sheath circumscribing at least a portion of the tube; and
a septal occluder retained within the sheath, the septal occluder comprising:
a structural framework comprising a plurality of struts interconnected to form a mesh, wherein the structural framework comprises:
a first end section;
a second end section; and
a cylindrical section interposed between the first end section and the second end section, wherein the cylindrical section extends along a centerline axis to define a bore, and wherein the first end section and the second end section extend radially outward relative to the centerline axis from the cylindrical section;
a flexible skin attached to the structural framework, wherein the flexible skin comprises a first membrane gate spanning the bore; and
a mechanical gate attached to the structural framework that is resiliently displaceable between a closed position spanning the bore and an open position in which the bore is at least partially unobstructed by the mechanical gate.

19. The system of claim 18, wherein the flexible skin includes a second membrane gate spanning the bore from the structural framework, and wherein the first membrane gate is closer to the first end section than the second end section, and wherein the second membrane gate is closer to the second end section than the first end section.

20. The system of claim 19, wherein the mechanical gate comprises:
a ring circumscribing the centerline axis;
a flap; and
a hinge member connecting the ring to the flap, wherein the flap is displaceable relative to the ring about the hinge member,
wherein the mechanical gate is disposed between the first membrane gate and the second membrane gate.
